(19) United States
(12) Patent Application Publication
(10) Pub. No.: US 2021/0008028 A1
(43) Pub. Date: Jan. 14, 2021

(54) COMPOSITIONS AND METHODS FOR TREATING DIARRHEAL DISEASES

(71) Applicant: VANESSA RESEARCH, INC., HAMDEN, CT (US)

(72) Inventors: Dmitry KRAVTSOV, Cheshire, CT (US); Ludmila I. KVOCHINA, Shelton, CT (US); Norman GRAY, Hamden, CT (US); Adrian Subia SAINZ, Navarra (ES)

(73) Assignee: VANESSA RESEARCH, INC., HAMDEN, CT (US)

(21) Appl. No.: 17/040,749

(22) PCT Filed: Mar. 23, 2019
(86) PCT No.: PCT/IB2019/052371

§ 371 Date: Sep. 23, 2020

Related U.S. Application Data
(60) Provisional application No. 62/647,622, filed on Mar. 23, 2018.

(51) Int. Cl. A61K 31/353; A61K 36/45; A61K 36/736; A61P 1/12

(52) U.S. Cl. CPC A61K 31/353; A61P 1/12; A61K 36/736; A61K 36/45

(57) ABSTRACT

Provided is a composition for the treatment of diarrheal disease, comprising a polyphenol-rich extract of a mixture of 80-20% by weight of blueberry or bilberry from Vaccinium cyanococcus spp., Vaccinium myrtillis spp., or both; and 20-80% by weight of sloe berry from Prunus spinosa spp. Other compositions, and methods of making and using the compositions for the treatment of diarrheal disease are disclosed.

### RELATED APPLICATIONS

This application claims the benefit of U.S. Application 62/647,622, filed Mar. 23, 2018, the entire contents of which are hereby incorporated by reference.

### FIELD OF THE INVENTION

The invention relates to compositions for the treatment of diarrheal diseases, and methods for their use in treating diarrheal diseases. Particular compositions include polyphenol-rich berry extracts, and mixtures of certain polyphenols. Methods of making the compositions are also described.

### BACKGROUND

Diarrheal disease is a tremendous socioeconomic and medical burden on the world. The World Health Organization (WHO) reports that diarrheal disease is the second leading cause of death in children under five years of age. Each year diarrhea kills around 525,000 children under the age of five, with 1.7 billion cases of childhood diarrheal disease every year (WHO publication https://www.who.int/news-room/fact-sheets/detail/diarrhoeal-disease as accessed on Mar. 19, 2019).

With the paucity of the specific antidiarrheal treatments available on the market today, there is an unmet need for the development of safe and efficient anti-diarrheal medications.

### BRIEF DESCRIPTION OF THE FIGURES

FIG. 1 presents data showing that polyphenol-rich extract significantly reduced fluid accumulation elicited by CTx in a dose-dependent manner.

FIG. 2 presents data showing that polyphenol-rich extract significantly reduced fluid accumulation elicited by CTx in a dose-dependent manner.

FIG. 3 presents data showing that polyphenol-rich extract did not significantly reduce fluid accumulation elicited by CTx in a dose-dependent manner.

FIG. 4 presents data showing that polyphenol-rich extract did not significantly reduce fluid accumulation elicited by CTx in a dose-dependent manner.

FIG. 5 presents data showing results of mass-spectroscopy experiments indicating the content of specific monomers before and after hydrolysis are presented.

FIG. 6 presents data showing results of mass-spectroscopy experiments indicating the content of specific monomers before and after hydrolysis are presented.

FIG. 7 presents data showing results of mass-spectroscopy experiments indicating the content of specific monomers before and after hydrolysis are presented.

FIG. 8 presents data showing results of mass-spectroscopy experiments indicating the content of specific monomers before and after hydrolysis are presented.

FIG. 9 presents IC50 data of polyphenol from the present study.

FIG. 10 presents IC50 data of polyphenol from the present study.

FIG. 11 presents IC50 data of polyphenol from the present study.

FIG. 12 presents IC50 data of polyphenol from the present study.

FIG. 13 presents data showing an exemplary composition having a synergistic inhibitory effect on CTx-induced fluid secretion in the mouse intestine, reducing the mass of accumulated fluid (average mass accumulation) by 25%.

### BRIEF DESCRIPTION OF THE SEVERAL EMBODIMENTS

One embodiment provides a composition for the treatment of diarrheal disease, comprising a polyphenol-rich extract of a mixture of:

a) 80-20% by weight of blueberry or bilberry from Vaccinium cyanococcus spp., Vaccinium myrtillis spp., or both; and

b) 20-80% by weight of sloe berry from Prunus spinosa spp.

Another embodiment provides a method for making a polyphenol-rich extract, comprising:

1. mixing 50-70% alcohol with a blueberry, bilberry, sloeberry, or chokeberry, to form a mixture;

2. incubating the mixture at room temperature for a time ranging from 1 hour to 90 days while rotating daily, to form an incubated mixture;

3. filtering or centrifuging solid matter out of the incubated mixture, and collecting flow-through of the incubated mixture;

4. optionally, combine the flow-through from Step 3 with a berry powder, to form a second mixture;

5. optionally, incubating the second mixture for 1-3 hours at room temperature, to form a second incubated mixture;

6. optionally, filtering or centrifuging solid matter out of the second incubated mixture and collecting supernatant; and

7. evaporating alcohol under vacuum from the flow-through from step 3 or the supernatant from step 4, at a temperature ≤40° C., to a desired alcohol concentration, such as 20% or less, to form the polyphenol-rich extract.

Another embodiment provides a composition for the treatment of diarrheal disease, comprising at least 0.2% by weight of each of: cyanidin, delphinidin, epicatechin gallate or epigallocatechin gallate, salt thereof, or glycosylate thereof; and a pharmaceutically acceptable carrier or excipient.

Another embodiment provides a method for treating a subject suffering from a diarrheal disease, the method comprising administering any of the compositions described herein to the subject, to treat said subject.

### DETAILED DESCRIPTION OF THE SEVERAL EMBODIMENTS

One embodiment provides a polyphenol-rich extract composition comprising a mixture of a) blueberry (a.k.a. bilberry) e.g., Vaccinium cyanococcus, Vaccinium myrtillis spp., or both; b) sloe berry, e.g., Prunus spinosa spp.; and/or c) chokeberry Aronia melanocarpa spp.

One embodiment provides a composition comprising a mixture of epicatechin; epigallocatechin; cyanidin; and delphinidin. Each of the epicatechin, epigallocatechin, cyanidin, and delphinidin may independently be in any form, for example in glycosylated form (i.e. as L-rhamnose, D-glucose, glucorhamnose, galactose, fructose or arabinose) or aglycone form; as crystallized form; aqueous solution; alcoholic solution; salt such as chloride salt, gallic acid salt; or combination thereof.

One embodiment provides a method for treating a subject suffering from a diarrheal disease, the method including administering either of the aforementioned compositions to the subject suffering from a diarrheal disease, such as cholera, travelers' diarrhea, E. coli diarrhea, Vibrio cholera and other Vibrio diarrheas, Clostridium difficile diarrhea, Klebsiella pneumoniae diarrhea, Rotovirus diarrhea, Adenovirus diarrhea, Parvovirus diarrhea, Norwalk virus (Norovirus) diarrhea, Giardia diarrhea, Astrovirus diarrhea, Calicivirus diarrhea, Shigella diarrhea, Salmonella diarrhea, Staphylococcus, Campylobacter, Yersinia, Aeromonas, Pseudomonas, Torovirus, Coronavirus, Picobirnavirus, Pestivirus, AIDS-related diarrhea; inflammatory diarrheal disorders, such as Inflammatory bowel disease, Crohn's disease, irritable bowel syndrome. The diarrhea may be induced by or exacerbated by toxin, such as cholera toxin, heat-stable enterotoxin, heat-liable enterotoxin, shiga-toxins, cytotoxins etc. In one embodiment, the diarrheal disease is cholera, induced or exacerbated by cholera toxin.

One embodiment provides a method for extraction of polyphenols from pre-mixed freeze-dried blueberry (a.k.a. bilberry), sloe berry, or chokeberry powder, where the mass fraction of each of the berry powders can independently vary from 1% to 99%; Where the alcohol used for extraction is ethyl alcohol in the concentration from 0% to 99%; where the alcohol is further removed from the resulting extract, which can remain a liquid or be desiccated to dryness by a suitable method, such as thin film-drying; where the extraction, removal of alcohol and dehydration are carried out while being protected from light; where all the steps are carried out at temperatures not exceeding 40 degree Celsius.

In one embodiment, the extraction may be carried out as follows:
1. Mix 50-70% alcohol (ethyl alcohol) with a berry mixture, for example a mixture of fresh or frozen blueberries and sloe berries at a desired w/w ratio. Alternatively, mix 50-70% alcohol (ethyl alcohol) with a dried berry, powdered berry, or freeze-dried berry powder mixture, for example a mixture of blueberry and sloe berry at a desired w/w ratio.
2. Incubate for 1 hour to 90 days while rotating daily room temperature.
3. Filter or centrifuge the solid matter out and collect flow-through.
4. Optionally, combine the flow-through from Step 3 with a berry powder, such as a freeze-dried berry powder, at a desired w/v ratio grams of powder to 10 ml of the flow-through from step 3.
5. Incubate for 1-3 hours at room temperature.
6. Filter solid matter and collect supernatant.
7. Evaporate alcohol under vacuum. Temperature of the supernatant should not exceed 40° C. during the evaporation. Continue evaporation until alcohol concentration is reduced to below 5%.
8. Optionally, measure polyphenolic content and adjust it to desired (for example, 15 mg/ml) by changing the amount of powder, evaporation time, to prepare the resultant polyphenol-rich extract.

In another embodiment, the extraction may be carried out as follows:
1. Mix 50-70% alcohol (ethyl alcohol) with sloe berries at 1:1 w/w ratio.
2. Incubate for 1 hour to 90 days while rotating daily room temperature.
3. Filter or centrifuge the solid matter out and collect flow-through.
4. Combine the flow-through with added blueberry powder at 3:10 w/v ratio grams of powder to 10 ml of the flow-through from step 3.
5. Incubate for 2 hours at room temperature.
6. Filter solid matter and collect supernatant.
7. Evaporate alcohol under vacuum. Temperature of the supernatant should not exceed 40 C during the evaporation. Continue evaporation until alcohol concentration is reduced to below 5%.
8. Optionally, measure polyphenolic content and adjust it to desired (for example, 15 mg/ml) by changing the amount of powder, evaporation time, if desired, to prepare the resultant polyphenol-rich extract.

In another embodiment, the extraction may be carried out as follows:
1. Mix 50% alcohol (preferably beet alcohol) with sloe berries at 1:1 w/w ratio.
2. Incubate for 90 days while rotating daily.
3. Filter the solid matter out and collect flow-through.
4. Combine the flow-through with blueberry powder at 3:10 w/w ratio
5. grams of powder to 10 ml of the flow-through from step 3 ratio.
6. Incubate for 2 hours at room temperature.
7. Filter solid matter and collect supernatant.
8. Evaporate alcohol under vacuum. Temperature of the supernatant should not exceed 40 C during the evaporation. Continue evaporation until alcohol concentration is reduced to below 5%.
9. Measure polyphenolic content and adjust it to desired (for example, 15 mg/ml) by changing the amount of powder, evaporation time, to prepare the resultant polyphenol-rich extract.

The concentration of the alcohol for mixing with the blueberry, bilberry, sloeberry, or chokeberry (whether powder or fresh/frozen, etc.) is not particularly limiting, but is desirably a 50-70% aqueous alcohol solution. This range includes all values and subranges therebetween, including 50, 55, 60, 65, and 70% alcohol (aq). Or 100 to 140 “proof” alcohol, in some cases. The alcohol is preferably suitable for pharmaceutical applications, food and medicine grade applications, and similar, but is not a requirement. The alcohol may be certified organic, if desired. Examples include ethyl alcohol, vegetable alcohol, beet alcohol, or combination thereof.

In embodiments, the composition independently includes 80-20% by weight of blueberry or bilberry from Vaccinium cyanococcus spp., Vaccinium myrtillis spp., or both. This range includes all values and subranges therebetween, including 80, 75, 70, 65, 60, 55, 50, 45, 40, 35, 30, 25, and 20% by weight, or any range therein, based on the weight of the polyphenol-rich extract.

In embodiments, the composition independently includes 80-20% by weight of blueberry from Vaccinium cyanococcus spp., Vaccinium myrtillis spp., or both. This range includes all values and subranges therebetween, including 80, 75, 70, 65, 60, 55, 50, 45, 40, 35, 30, 25, and 20% by weight, or any range therein, based on the weight of the polyphenol-rich extract.

In embodiments, the composition independently includes 20-80% by weight of sloe berry from Prunus spinosa spp. This range includes all values and subranges therebetween, including 20, 25, 30, 35, 40, 45, 50, 55, 60, 65, 70, 75, and 80% by weight, or any range therein, based on the weight of the polyphenol-rich extract.

In embodiments, the composition includes 80-20% by weight of blueberry or bilberry from Vaccinium cyanococcus spp., Vaccinium myrtillis spp., or both; and 20-80% by weight of sloe berry from Prunus spinosa spp. Here, these respective ranges include all values and subranges therebetween, as noted elsewhere herein.

In embodiments, the composition may further include chokeberry from Aronia melanocarpa spp. Chokeberry may, if desired, be present at 0.1-40% by weight. This range includes all values and subranges therebetween, including 0.1, 0.5, 1, 2, 3, 4, 5, 10, 15, 20, 25, 30, 35, and 40% by weight, based on the weight of the polyphenol-rich extract.

For example, the mixture (or extract) may include 70-30% (or 70, 60, 50, 40, 30%) by weight of blueberry or bilberry; 60-40% (or 60, 50, 40%) by weight of blueberry or bilberry; or 50:50 weight ratio of blueberry or bilberry:sloeberry.

In other embodiments, the weight ratio of blueberry or bilberry:sloeberry in the mixture or polyphenol-rich extract ranges from 80-20:20-80, which respective ranges include all values and subranges therebetween. For example, ratios of 80:20, 21, 22, 24, 26, 28, 30, 34, 40, 42, 44, 46, 50, 52, 54, 60, 68, 70, 72, 76, 80:20 are contemplated.

In embodiments, the berry, e.g., blueberry, bilberry, sloeberry, or chokeberry in the mixture is in the form of berry powder (powdered berry), freeze-dried powder, or combination thereof.

In embodiments, the berry, e.g., blueberry, bilberry, sloeberry, or chokeberry in the mixture is in the form of fresh, frozen, dried, or freeze-dried berries, or combination thereof.

The first incubation of the mixing alcohol and the berry mixture is desirably carried out for 1 hour to 90 days, preferably while rotating, shaking, or stirring daily. The time range includes all values and subranges therebetween, including 1, 2, 3, 4, 5, 6, 7, 8, 9, 10, 11, 12, 18 hours, 1, 2, 3, 4, 5, 6, 7, 8, 9, 10, 20, 40, 50, 60, 70, 80, 90 days.

The incubation of the mixing alcohol and the berry mixture is desirably carried out at room temperature, or about 25° C.

The incubation of the mixing alcohol and the berry mixture is desirably carried out in the dark, for example, not exposed to visible or UV radiation.

After the first incubation, the solid matter may be separated by filtration or centrifugation; and the flow-through is collected. In embodiments, unless additional powder is desired to be added, the extract at this stage can be the polyphenol-rich extract.

Otherwise, the aforementioned flow-through can be further combined with a berry powder, such as a freeze-dried berry powder, at a desired w/v ratio. For example, berry powder may be added to the flow-through in an amount equivalent to 1-5:10 w/v ratio grams of powder to ml of the flow-through. This range includes all values and subranges therebetween, including 1, 1.5, 2, 2.5, 3, 3.5, 4, 4.5, and 5:10 w/v. Obviously, the equivalent amounts may be scaled up for production as desired.

If additional berry powder is added, a second incubation for 1-3 hours may be carried out at room temperature, if desired. And afterwards, solid matter is collected by filtration or centrifugation, and the supernatant is collected.

After the first or second incubation, alcohol is evaporated under vacuum. Temperature of the supernatant should not exceed 40° C. during the evaporation. Continue evaporation until alcohol concentration is reduced to below 20, 15, 10, or 5%.

The polyphenolic concentration of the polyphenol-rich extract may be measured as described herein. It may be adjusted by addition, dilution, or further evaporation as desired. For example, the polyphenol content of the polyphenol-rich extract may suitably range from 0.2-60 mg/ml, which range includes all values and subranges therbetween, including 0.2, 0.5, 1, 2, 3, 4, 5, 7, 9, 10, 15, 20, 25, 30, 35, 40, 45, 50, 55, and 60 mg/ml.

In embodiments, the polyphenol-rich extract includes one or more of cyanidin, delphinidin, epicatechin gallate or epigallocatechin gallate, salt thereof, glycosylate thereof, or combination thereof. In embodiments, the polyphenol-rich extract includes each of cyanidin, delphinidin, epicatechin gallate, and epigallocatechin gallate, salt thereof, glycosylate thereof, or combination thereof.

In one embodiment, the method includes contacting the berry powders with ethanol, incubating at 40° C. for a time, then removing the alcohol by vacuum distillation at about 40-50 mbar at 40° C., to evaporate to a solid polyphenol-rich extract.

We tested an extract of polyphenols from a combination of common edible berry species for the potential of neutralizing the secretory diarrheal disease and with a goal of creating a natural, safe and efficacious antidiarrheal compound.

The combination included blueberry (also known as bilberry in some territories) from the Vaccinium cyanococcus and Vaccinium myrtillis plant species, sloe berry from Prunus spinosa plant species and/or chokeberry from Aronia melanocarpa plant species. All aforementioned berry species are rich natural sources of polyphenols from the flavan-3-ols and gallotannins chemical classes. Our tests focused on the elucidation of potential benefits of the extract as applicable to the inhibition of intestinal secretion.

In embodiments, a composition for the treatment of diarrheal disease is provided, comprising at least 0.2% by weight of each of: cyanidin, delphinidin, epicatechin gallate or epigallocatechin gallate, salt thereof, or glycosylate thereof; and a pharmaceutically acceptable carrier or excipient. This range includes all values and subranges therebetween, including 0.2, 0.4, 0.6, 0.8, 1, 2, 3, 4, 5, 6, 7, 8, 9, 10, 15, 20, 22, 25, 27, 30, 33, 35, 37, 39, 40, 44, 45, 50, 55, 60, 65, 70, 75, 80, 85, 90, 95, 98, 99, 99.1, and 99.4% by weight, independently for each of cyanidin, delphinidin, epicatechin gallate or epigallocatechin gallate, salt thereof, or glycosylate thereof.

In embodiments, the composition may have a weight ratio for cyanidin:delphinidin:epicatechin gallate:epigallocatechin gallate is 300-700: 100-30:0.5-2:200-50. These ranges include all respective values and subranges therebetween, including 300, 350, 400, 450, 500, 550, 600, 650, 700: 100, 90, 80, 70, 60, 50, 40, 30:0.5, 0.7, 0.9, 1, 1.5, 2:200, 175, 150, 125, 100, 75, 50.

In one embodiment, the weight ratio of cyanidin:delphinidin:epicatechin gallate:epigallocatechin gallate is 400-600:90-50:0.75-1.5:175-100.

In another embodiment, the weight ratio of cyanidin:delphinidin:epicatechin gallate:epigallocatechin gallate is 500:70:1:150.

Here, the weight ratios for cyanidin:delphinidin:epicatechin gallate:epigallocatechin gallate is considered to hold for any one or more of the salts, glycosylated form, or combination thereof.

Any of the epicatechin, epigallocatechin, cyanidin, and delphinidin may independently be in glycosylated form, L-rhamnose, D-glucose, glucorhamnose, galactose, fructose or arabinose form, aglycone form, crystallized form, aqueous solution, alcoholic solution, salt, chloride salt, gallic acid salt, or combination thereof.

Preferred route of administration is oral, but other administration routes are contemplated, including rectal, sublingual or buccal, or a combination thereof. Suggested dosing schedule is 3-4 times per day.

Indications include any diarrheal disorders where the secretory component is present as a part of the pathogenesis of the disease. Proposed subjects are human and non-human animals of all age groups, including pediatric population. Disease spectrum is the bacterial, viral or parasitic infectious diarrheas, exemplified in the experiments by administration of cholera toxin as causative agent for diarrhea. Further, inflammatory diarrheal disorders such as inflammatory bowel disease, Crohn's disease and irritable bowel syndrome may clinically benefit from the treatment with the extract.

Extract may be administered in the pure form or could be further formulated into an oral liquid by mixing with water, glycerol and other excipients, such as preservatives (i.e. sodium benzoate, potassium sorbate, and other acceptable equivalents); taste modifiers (sugars, i.e. sorbitol, erythritol, glucose, fructose etc.; extracts of other plants, i.e. cherry, orange, strawberry etc.); consistency modifiers, such as guar gum, xanthan gum, methylcellulose) and other excipients as per the current standards in field of pharmacological formulations.

### EXAMPLES

The following examples are provided for illustration purposes and for better understanding of some of the benefits of the present invention, and is not intended to be limiting unless otherwise specified.

Extraction experiments were conducted with the use of several common solvents known to elute and extract polyphenols. We tested acetone, methanol, ethanol and water, as well as various combinations of thereof, as extractants. The blueberries were purchased in the form of fresh, frozen (StopandShop, Hamden, Conn., USA), or dried-powdered fruits (NutriSeed, London, UK). Sloe berries and powder were purchased from DZ Licores (DZ Licores, Dicsatillo, Spain). Chokeberries were purchased from Amazon.com (Amazon, Seattle, Wash., USA). After extraction, the total polyphenolic content in the mixture was determined by well-characterized Folin-Ciocalteu (FC) colorimetric method. The highest yield of polyphenols was observed when using the following two methods.

### Example 1—Method A

Fresh or freshly frozen fruit mixtures were combined with 96% beet alcohol (DZ Licores) as 50/50 v/v ratio. Fruit mixture consisted of a pre-weighed combination of 50% sloe berries, 49% blueberries and 1% chokeberries as w/w ratio. After adding alcohol, the extraction mixture was incubated in the dark with periodic agitation for 90 days, followed by separation and collection of the liquid phase. Residual alcohol was evaporated from the extract, and the aqueous phase was studied for the concentration of the total polyphenols by FC method. Water in the reaction came from the berries since it is the main constituent of fresh and freshly frozen fruits, comprising up to 90% of the total weight of the fruits.

Such extraction method yielded total polyphenol concentrations in the mixtures up to 15 mg/ml of extract.

It was found that the main factors negatively affecting the yield were exposure to the light and heating the mixture to above 40 degree Celsius.

Similar extractions with acetone or methanol were less efficient.

To further increase the yield of polyphenols, we hypothesized that the limiting factors for the extraction were a) water content in the fresh fruits and b) specific percentage of the ethyl alcohol in the reaction.

### Example 2—Method B

To improve the control over the amount of water in the extraction reaction we reverted to the use of dried powdered fruits. Of all powders tested, freeze-dried berries demonstrated superiority as to the yield of the polyphenols in the extracts. Several w/w ratios of berry powders were tested, and the 50/50 w/w ratio of blueberry and sloe berry powders was found as the most efficient overall, however, the weight percent of each of the berries can vary significantly from 5 to 95% without much loss of the polyphenol concentration in the resulting extract. An aqueous ethyl alcohol (food grade) was used as extractant in the range of concentration from 95% alcohol to pure water. Extraction reactions were set up in the 1:3 v/v ratio of berry powder to extractant. Reactions were conducted in the dark for 1-2 hours at room temperature with continuous agitation. Further increase in the reaction time had diminishing return on the reaction efficiency. After incubation, liquid phase was separated from the reactions and collected. Alcohol was removed from the liquid phase by evaporation. Reaction temperature was controlled during all phases in order not to exceed 40° C. The yield of polyphenols (in mg/ml) was measured by FC method.

This extraction method significantly increased the concentration of polyphenols to 30 mg/ml of the extract. Extraction efficiency increased with increasing the alcohol percentage, reaching the peak between 50% and 70% v/v alcohol, after which efficiency began declining again, reaching the minimum at 95% alcohol.

### Example 3—Mouse Model with Polyphenol Extract

The extracts were further subjected to the testing in mouse models of secretory diarrhea as follows:

The extract obtained from Method B with the 1:1 w/w mixture of blueberry and sloe berry freeze-dried powders was subjected to testing in the intestinal secretion mouse models as described below. All batches of extracts that we used in the experiments were normalized to 15 mg/ml total polyphenol concentration, and are further referred to as “polyphenol extract”.

Goal: Assess anti-secretory effect of polyphenol extract in the cholera-toxin (CTx) stimulated intestinal fluid secretion mouse model.

Mouse: adult C57BL6 strain

Animals were weighed and anesthetized with Avertin (Sigma-Aldrich, Saint Louise, Mo., USA) (250 mg/kg IP induction dose f.b. 2.6 mg IP every 30-45 minutes as needed for maintenance of anesthesia). Abdomen was opened. Small, and part of the large intestine were identified. 1.5-3 cm loops were ligated in the proximal jejunum beginning immediately downstream of Treitz ligament. Loops were separated by 1-2 cm of intervening small intestine. After ligation, proximal loop was injected with 100 μl of PBS (phosphate buffered saline) (loop 1), middle loop was injected with CTx solution in 100 μl PBS (Loop 2) and distal loop was injected with CTx and polyphenol extract (PP extract, diluted as described) solution in 100 μl of PBS (loop 3). Injected loops were photographed and carefully placed back into the abdominal cavity and then abdomen was closed with two sutures. After 4 to 6-hour incubation, animal was euthanized and entire length of small intestine, cecum and ascending transverse colon were removed as a single prep. Loops were excised, trimmed of fat, measured and weighted.

Results:

A. CTx 10 μg per loop (Sigma-Aldrich), polyphenol extract diluted 1:10 parts v/v (in PBS or H₂O as appropriate) per loop, 100 μl total, 4-5 hour incubation, n=3

1. PBS was absorbed from the loop 1, which returned in appearance to the appearance of empty intestine. Weight/length ratio was 3.77±0.66 mg/mm

2. CTx-stimulated loop 2 was visibly distended with fluid. Weight/length ratio was 9.5±2.23 mg/mm

3. CTx-stimulated polyphenol extract-treated loop 3 was dark purple in color, and considerably less distended then loop 2. Length was measured at 32 mm and weight at 0.1473 g. Weight/length ratio was 5.33±0.70 mg/mm

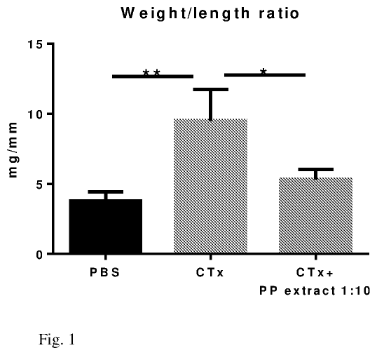

Statistical analysis was performed using one-way ANOVA Sidak's test corrected for multiple comparisons. Multiplicity-adjusted p-values were calculated for PBS to CTx (p=0.0074), CTx to CTx+PP extract (p=0.0325) and PBS to CTx+ polyphenol extract (p=0.5276) treatment groups. N=3 for each treatment group (FIG. 1).

B. CTx 1 μg (Cayman chemical, Ann Arbor, Mich., USA) per loop, polyphenol extract 1:100 per loop in 100 μl total volume, 5.5 to 6 hours incubation, n=3

1. PBS was absorbed from the loop 1, which returned in appearance to the appearance of empty intestine. Weight/length ratio was 3.128±0.190 mg/mm

2. CTx-stimulated loop 2 was visibly distended with fluid. Weight/length ratio was 7.316±1.089 mg/mm

3. CTx-stimulated polyphenol extract-treated loop 3 was dark purple in color, and considerably less distended then loop #2. Weight/length ratio was 3.804±0.743 mg/mm

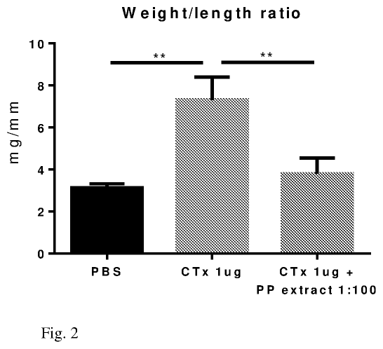

Statistical analysis was performed using one-way ANOVA Sidak's test corrected for multiple comparisons. Multiplicity-adjusted p-values were calculated for PBS to CTx (p=0.0011) and CTx to CTx+ polyphenol extract (p=0.0028) treatment groups. N=3 for each treatment group (FIG. 2).

C. CTx 1 μg per loop, polyphenol extract 1:1000 per loop in 100 μl total volume, 4 to 6 hours incubation.

1. PBS was absorbed from the loop 1, which returned in appearance to the appearance of empty intestine. Weight/length ratio was 3.515±0.994 mg/mm

2. CTx-stimulated loop 2 was visibly distended with fluid. Weight/length ratio was 7.015±0.979 mg/mm

3. CTx-stimulated polyphenol extract-treated loop 3 was dark purple in color, and considerably less distended then loop #2. Weight/length ratio was 6.131±2.044 mg/mm

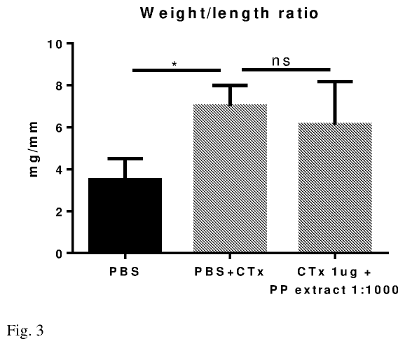

Statistical analysis was performed using one-way ANOVA Sidak's test corrected for multiple comparisons. Multiplicity-adjusted p-values were calculated for PBS to CTx (p=0.018) and CTx to CTx+ polyphenol extract (p=0.644-) treatment groups. N=4 for each treatment group (FIG. 3).

D. CTx 1 μg per loop, polyphenol extract 1:10000 per loop in 100 μl total volume, 4 to 6 hours incubation.

1. PBS was absorbed from the loop 1, which returned in appearance to the appearance of empty intestine. Weight/length ratio was 3.147±0.0.069 mg/mm

2. CTx-stimulated loop 2 was visibly distended with fluid. Weight/length ratio was 7.602±2.134 mg/mm

3. CTx-stimulated polyphenol extract-treated loop 3 was same or more as distended as the CTx-stimulated loop 2. Weight/length ratio was 9.571±5.495 mg/mm

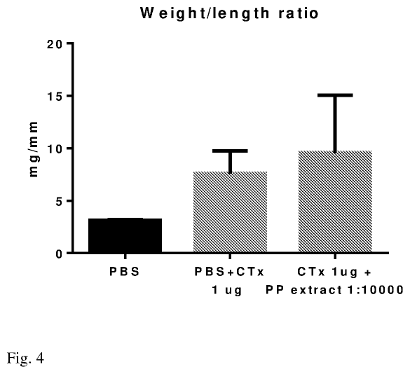

Statistical analysis was performed using one-way ANOVA Sidak's test corrected for multiple comparisons. Multiplicity-adjusted p-values were calculated for PBS to CTx (p=0.295) and CTx to CTx+ polyphenol extract (p=0.755) treatment groups. N=3 for each treatment group (FIG. 4), yielding no significant difference between the treated and untreated groups.

Conclusion: Polyphenol extract significantly reduced fluid accumulation elicited by CTx (FIG. 1-4) in a dose-dependent manner Since CTx is inducing secretion via upregulation of cAMP-dependent intestinal chloride transport, PP extract acted as an anti-secretory antidiarrheal capable of antagonizing the effect of CTx on the mouse proximal small intestine. Effect of PP extract diminishes as dilution approaches 1:1000 parts v/v (FIG. 3) and disappears completely at 1:10000 dilution (FIG. 4).

The results from Example 3 indicate a suitable range of concentrations of the polyphenol extract from 60 microgram/kilogram to 600 milligram/kilogram in the mouse for neutralizing the effect of cholera toxin on the intestinal secretion. This range includes all values and subranges therebetween, including 60, 70, 80, 90, 100, 120, 140, 160, 180, 200, 300, 400, 500, 600 mg/kg in the mouse, scalable up to humans if desired.

Recalculations of the dose to human subjects suggests the working range from 5 microgram/kilogram to 50 milligram/kilogram of the polyphenol extract. This range includes all values and subranges therebetween, including 5, 6, 7, 8, 9, 10, 15, 20, 25, 30, 35, 40, 45, 50 mg/kg in the human. Toxic effects are not expected until the single dose of 10 gram/kg.

### Example 4—Chemical Characterization of Extract

We chemically characterized the extract using liquid chromatography coupled with mass-spectrometry.

It was found that cyanidin, delphinidin, epicatechin gallate, and epigallocatechin gallate were the predominant forms of the flavonoids and gallotannins in the extract. Their structures are below.

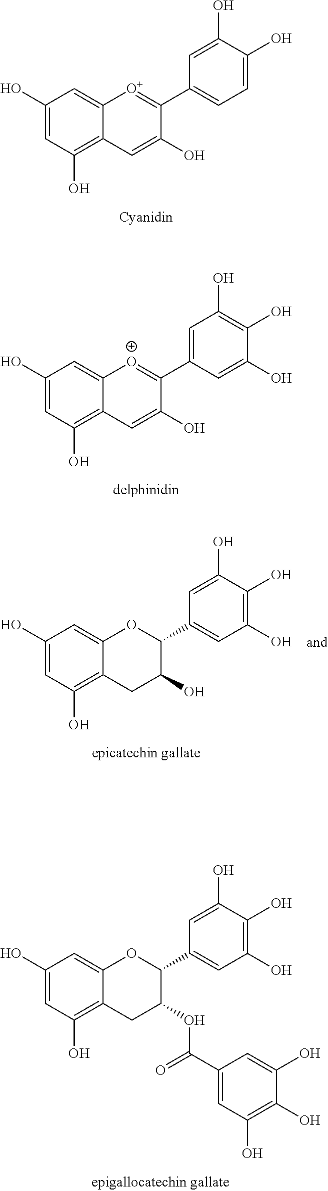 [SMILES: Oc1cc(O)c2cc(O)c(-c3ccc(O)c(O)c3)[o+]c2c1 ; Oc1cc(O)c2cc(O)c(-c3cc(O)c(O)c(O)c3)[o+]c2c1 ; Oc1cc(O)c2c(c1)O[C@H](c1cc(O)c(O)c(O)c1)[C@@H](O)C2 ; O=C(O[C@@H]1Cc2c(O)cc(O)cc2O[C@@H]1c1cc(O)c(O)c(O)c1)c1cc(O)c(O)c(O)c1]

We also addressed a critical question of the relative ratio of polymeric vs. monomeric chemical forms in the extract by performing acid hydrolysis of both the extracts as well as individual species of V. cyanococcus, P. spinosa and A. melanocarpa.

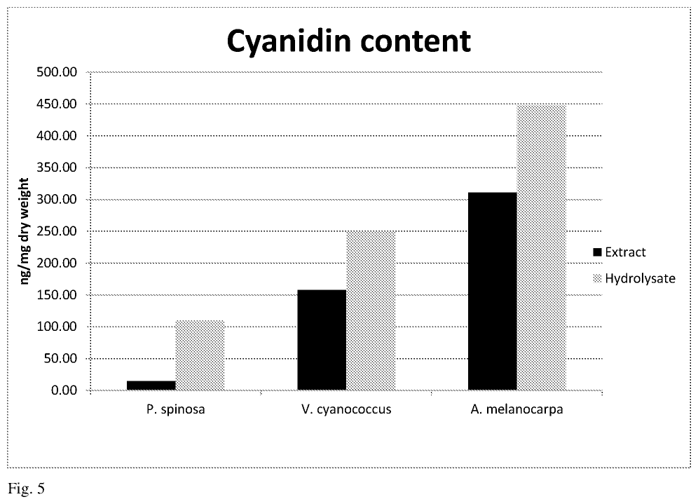

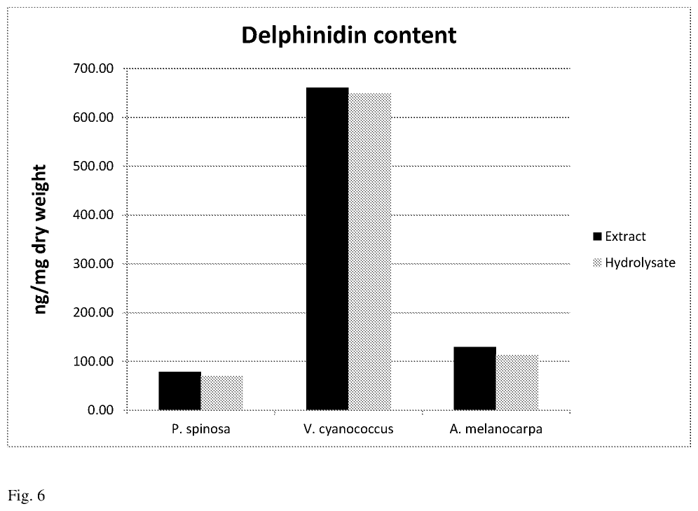

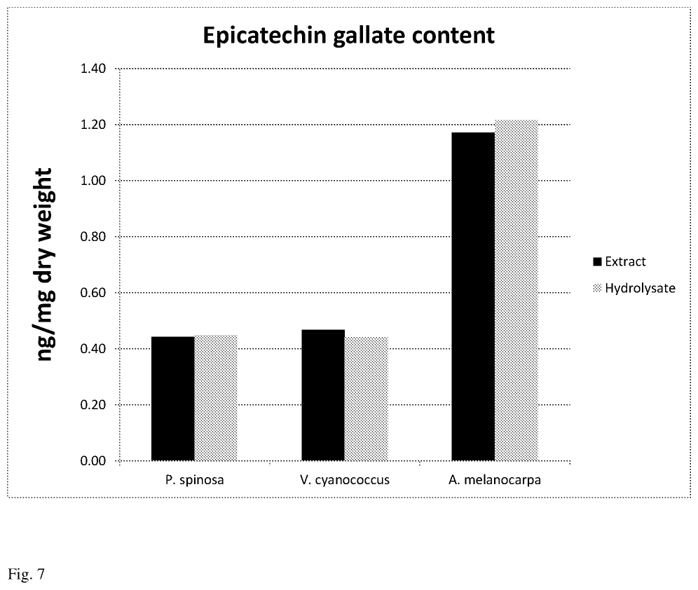

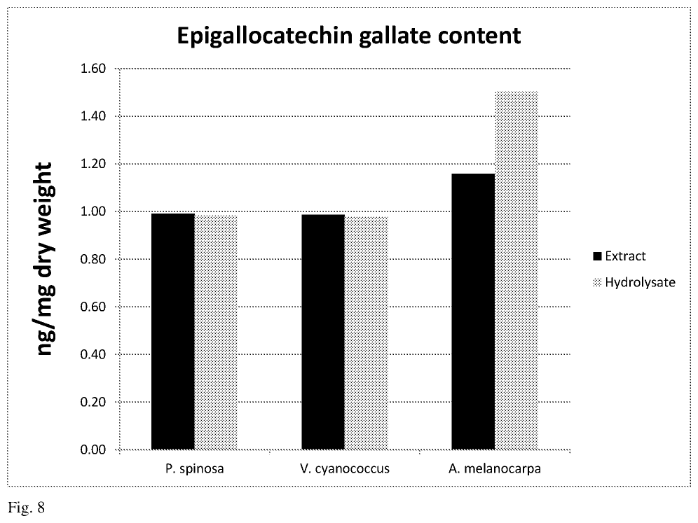

The results of mass-spectrometry of the polyphenol extract indicated that cyanidin, delphinidin, epicatechin gallate and epigallocatechin gallate are mostly monomeric in the materials that we've tested. The results of the MS experiments indicating the content of specific monomers before and after hydrolysis are presented in the FIG. 5-8.

In embodiments, the cyanidin, delphinidin, epicatechin gallate and epigallocatechin gallate are each independently monomeric, substantially entirely monomeric, or 99, 95, 90, 80, 70, 60, 50, 40, 30, 20, 10%, or less monomeric (polymeric).

### Example 5—Testing of Cyanidin, Delphinidin, Epicatechin Gallate and Epigallocatechin Gallate in the Mouse Model

Following identification, individual reagents of cyanidin, delphinidin, epicatechin gallate and epigallocatechin gallate were acquired from ChromaDex (Chromadex, Irvine, Calif., USA) and tested in the same cholera-toxin induced closed-loop mouse diarrheal model described above. We sought and resolved the IC⁵⁰ concentration of each individual component as relevant for the treatment of cholera-toxin induced intestinal secretion.

Further, we observed a synergistic effect of a combination of the monomeric forms of cyanidin, delphinidin, epicatechin gallate and epigallocatechin gallate, which was unexpected and surprising.

Goal: Demonstrate anti-secretory effect of polyphenols in the cholera-toxin (CTx) stimulated intestinal fluid secretion model.

Method

Mice (19-35 g) were fasted for 24-48 h, weighed and anesthetized with Avertin Sigma-Aldrich, St. Louise, Mo., USA) (250 mg/kg IP induction dose f.b. 2.6 mg IP every 30-45 minutes as needed for maintenance of anesthesia). Body temperature was maintained at 37-38° C. using a heating pad.

Abdomen was opened. Small, and part of the large intestine were identified. 2-4 cm loops were ligated in the proximal jejunum beginning immediately downstream of Treitz ligament. Loops were separated by 1-2 cm of intervening small intestine. After ligation, one loop was injected with 100 μl of PBS, second loop was injected with CTx solution (Cayman chemicals, 1 μg in 100 μl PBS and third loop was injected with CTx and polyphenol solution (Chromadex, Irvine, Calif., USA) in 100 μl of PBS. Injected loops were photographed and carefully placed back into the abdominal cavity and then abdomen was closed with two sutures. After 4 to 6-hour incubation, animal was euthanized and entire length of small intestine, cecum and ascending transverse colon were removed as a single prep. Loops were excised, trimmed of fat, measured and weighted. Mice were euthanized by cervical dislocation or an overdose injection of Avertin. All animal protocols were approved by the Laboratory Animal Ethical Committee of Vanessa Research Inc. The loop weight/length ratio was calculated.

Flavan-3-ols and gallotannins stock solutions (stock dilutions (stored at −20 C)) and working dilutions (PBS) were designated and prepared as follows:

F1A—Cyanidin chloride (Chromadex, ASB-00003955-005 Lot #00003955-041) 5 mg dissolved in 1 ml methanol (Sigma-Aldrich, Saint Louise, Mo., USA) to 5 mg/ml stock. Original concentration used in experiments 0.05 mg/ml. Serial dilutions from 1:10 to 1:100,000 with increments of 1:10 were prepared for experiments.

F1B—Delphinidin chloride (Chromadex, ASB-00004125-001 Lot #00004125-504) 1 mg dissolved in 143 ul methanol to 7 mg/ml stock. Original concentration used in experiments 0.07 mg/ml. Serial dilutions from 1:10 to 1:100,000 with increments of 1:10 were prepared for experiments.

F1C—Epicatechin gallate (Chromadex, ASB-00005135-005 Lot #00005135-523) 5 mg dissolved in 1 ml methanol to 5 mg/ml stock. Original concentration used in experiments 0.001 mg/ml (1:50 dilution of 5 mg/ml stock). Serial dilutions from 1:10 to 1:100,000 with increments of 1:10 were prepared for experiments.

F1D—Epigallocatechin gallate (Chromadex, ASB-00005150-005 Lot #00005150-008) 5 mg dissolved in 1 ml methanol to 5 mg/ml stock. Original concentration used in experiments 0.0015 mg/ml (1:33.4 of 5 mg/ml stock). Serial dilutions from 1:10 to 1:100,000 with increments of 1:10 were prepared for experiments.

Results

1. PBS was absorbed from the loop, which returned in appearance to the appearance of empty intestine. Weight/length ratio was 2.52±0.61 mg/mm

2. CTx-stimulated loop was visibly distended with fluid. Weight/length ratio was 10.14±2.19 mg/mm

3. CTx-stimulated polyphenol-treated loop demonstrated reduction in the weight/length ratio (mg/mm) depending on the treatment concentration/dilution

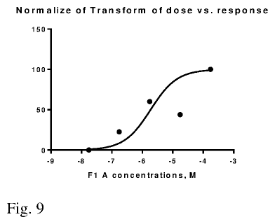

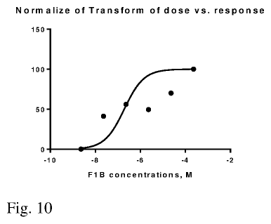

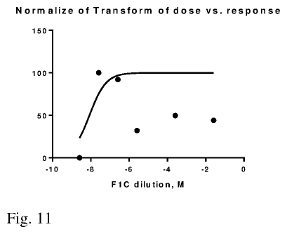

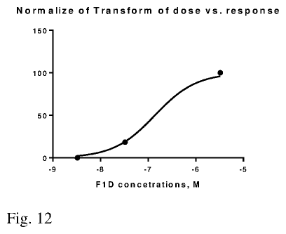

4. IC50 of each polyphenol used in a study F1A, F1B, and F1B EC50 falls in to nM-μM range (1×10^{-7 to -5}) and F1C IC50 falls in to nM range (1×10⁻⁸) as shown in the FIG. 9-12. The x-axes of FIGS. 9-12 should be read as 1×10^{nth}, where nth is the x-axis number.

The following dilutions of individual compound did not have any effect on the CTx-induced fluid secretion:

TABLE 1

| Compound | Dilution | Concentration, mg/ml |
|---|---|---|
| F1A | 1:10,000 | 5 × 10⁻⁶ |
| F1B | 1:100,000 | 7 × 10⁻⁸ |
| F1C | 1:100,000 | 1 × 10⁻⁸ |
| F1D | 1:1,000 | 1 × 10⁻⁶ |

However, in order to confirm or exclude the idea of synergism between multiple polyphenols in exertion of the anti-secretory effect following induction of fluid secretion with CTx, we attempted a treatment with a combination of all 4 polyphenols in the concentrations as indicated in Table 1. The ratio of concentrations (mg/ml) of individual members (cyaniding:delphinidin:epicatechin gallate:epigallocatechin gallate) in the combination was derived from Table 1 as 500:70:1:150 (weight ratio) accordingly.

Data analysis was done as follows: The formula was used as adapted from Yamamoto K., 1979:

Mass accumulation=Tₘ−(Cₘ/C_{L})×T_{L}

Tₘ=mass (mg) of Treated or untreated loop

Cₘ=mass (mg) of control loop

C_{L}=length (cm) of control loop

T_{L}=length (cm) of treated or untreated loop

After determining fluid accumulation in treated and untreated samples, a percent reduction in mass was performed using the mass obtained from the previous formula

(Uₘ−Cₘ)−(Tₘ−Cₘ)/(Uₘ−Cₘ)

Uₘ=mass accumulation of untreated loop (Ctx loop)

Cₘ=mass accumulation of control loop

Tₘ=mass accumulation of treated loop (Drug+Ctx)

*Note that this formula uses mass accumulation solved from the Yamamoto equation and not mass of the loop found experimentally*

5. The certain variables were removed based on the Exclusion Criteria for Data:

a. If Ctx loop was not greater than 7.00. Based on Ctx-Ctx loop experiment results

i. 7.00 is the lower bound of the mean value for combined proximal and distal CTx induced fluid accumulation

ii. Only the lower bound was used to differentiate the data because some mice were found to be low or non-responders to CTx. These low or non-responders are not characteristic of the population being targeted b/c they do not develop intestinal secretion, hence being inapplicable to the proposed mechanism of action. The upper bound, which was 11.84 mg/mm, was not used to exclude larger values, as the differentiation of medium to large responders was not necessary.

b. If mouse lived post-surgery less than 4.0 hours prior to excision of intestinal loops

c. In cases of significant leakage of intestinal loops occurred during trimming of adipose or connective tissue prior to measuring mass

d. If PBS loop was greater than 3.95 mg/mm

e. If loops of CTx and CTx with treatment less than 2 cm length

TABLE 2

| | F1ABCD | CTx | PBS |
|---|---|---|---|
| Average mass accumulation | 173.8782507 | 229.2355036 | 7.82859434 |
| (mg) | | | |
| St. Dev. P | 87.17346792 | 65.4037903 | 15.347998 |
| % Reduction of Drug from | 25.00249569 | | |
| Ctx | | | |
| P-value (Change of drug | | | 0.06387417 |
| from ctx) (mg) | | | |

The data demonstrated that a combination of cyanidin, delphinidin, epicatechin gallate and epigallocatechin gallate had an inhibitory effect on CTx-induced fluid secretion in the mouse intestine, reducing the mass of accumulated fluid (average mass accumulation) by 25%.

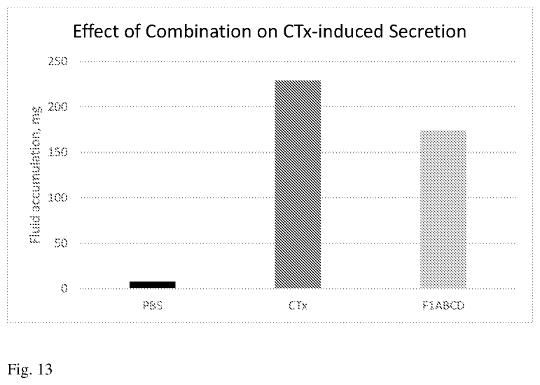

The results are further summarized in the FIG. 13

### Conclusions

1. Cholera toxin developed significant fluid accumulation in closed intestinal loop of alive animal (mouse) as a result of intensive secretion inside of loop.

2. All four polyphenols (cyanidin chloride, delphinidin chloride, epicatechin gallate, epigallocatechin gallate) significantly reduced fluid accumulation elicited by CTx in a dose-dependent manner Since CTx is inducing secretion via upregulation of cAMP-dependent intestinal chloride transport, polyphenols acted as an anti-secretory antidiarrheal capable of antagonizing the effect of CTx on the mouse proximal small intestine. Polyphenols effect diminishes proportionally to the dilution factor, indicating dose-response effect for the drug.

A combination of polyphenols has been shown to be more potent and effective against cholera toxin-stimulated development and progression of intestinal secretion than individual polyphenols. A mixture of four polyphenols, each in very low concentration, which did not show a therapeutic effect alone, was effective in inhibiting the secretion. As such, the polyphenol combination demonstrated higher treatment potency than individual polyphenols, indicating the synergism of the components. Polyphenols and mixtures of polyphenols should be considered as safe and effective approaches in cholera therapy and, possibly, prevention.

An unexpected and surprising observation from our experiments is that the combination of all four individual reagents (individual reagents of cyanidin, delphinidin, epicatechin gallate and epigallocatechin gallate) exerted an anti-secretory effect, whereas individual components alone in the same concentration demonstrated no observable effect on secretion. Thus, the combination of polyphenols is more efficient than any individual agent when used at the same concentration.

Polyphenols having —OH radicals in the positions R3′, R4′ and R5′ of the B-phenolic ring Table 3, as well as gallic acid residues may be particularly desirable for achievement of inhibitory effect on secretion. In addition, glycosylation at the R3 Table 3 is known to beneficially affect the water solubility of the molecule, allowing for easier formulations.

TABLE 3
Molecule numbering and glycosylation example

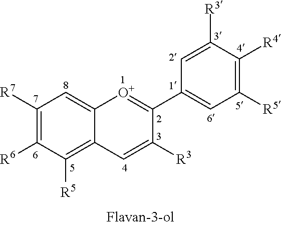

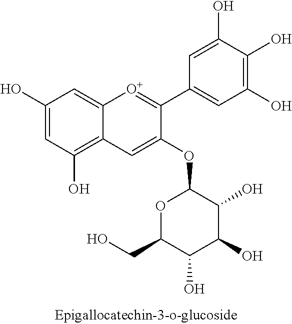 [SMILES: OC[C@H]1O[C@@H](Oc2cc3c(O)cc(O)cc3[o+]c2-c2cc(O)c(O)c(O)c2)[C@H](O)[C@@H](O)[C@@H]1O]

| | | R3′ | R4′ | R5′ | R3 | R5 | R6 | R7 |
|---|---|---|---|---|---|---|---|---|
| Cyanidin | Cy | OH | OH | H | OH | OH | H | OH |
| Delphinidin | Dp | OH | OH | OH | OH | OH | H | OH |
| Malvidin | Mv | OCH₃ | OH | OCH₃ | OH | OH | H | OH |
| Pelargonidin | Pg | H | OH | H | OH | OH | H | OH |
| Peonidin | Pn | OCH₃ | OH | H | OH | OH | H | OH |
| Petunidin | Pt | OH | OH | OCH₃ | OH | OH | H | OH |

Based on the results of our experiments, mass fraction of individual components in the mixture can vary from 0.2 to 99.4% depending on the potency of individual components.

Recalculations of the dose to human subjects suggests nano- to micromolar working IC₅₀ range, indicating very high efficacy of the mixture. Suggested dosage, as recalculated from mouse experiments, falls into the range from 1 microgram/kilogram to 10 milligram/kilogram. This range includes all values and subranges therebetween, including 1, 2, 3, 4, 5, 6, 7, 8, 9, 10, 20, 30, 50, 70, 90, 100, 200, 500, 700, 900 microgram, 1, 2, 3, 4, 5, 6, 7, 8, 9, 10 milligram/kilogram. Dose should not exceed 100 mg/kg due to the increased risk of toxicity.

1. A composition for the treatment of diarrheal disease, comprising a polyphenol-rich extract of a mixture of:
a) 80-20% by weight of blueberry or bilberry from Vaccinium cyanococcus spp., Vaccinium myrtillis spp., or both; and
b) 20-80% by weight of sloe berry from Prunus spinosa spp.

2. The composition of claim 1, wherein the mixture further comprises c) chokeberry from Aronia melanocarpa spp.

3. The composition of claim 1, wherein the mixture comprises 70-30% by weight of blueberry or bilberry.

4. The composition of claim 1, wherein the mixture comprises 60-40% by weight of blueberry or bilberry.

5. The composition of claim 1, wherein the mixture comprises a 50:50 weight ratio of blueberry or bilberry:sloeberry.

6. The composition of claim 1, wherein one or more of the blueberry, bilberry, sloeberry, or chokeberry in said mixture is in the form of powder.

7. The composition of claim 1, wherein one or more of the blueberry, bilberry, sloeberry, or chokeberry in said mixture is in the form of freeze-dried powder.

8. The composition of claim 1, wherein one or more of the blueberry, bilberry, sloeberry, or chokeberry in said mixture is in the form of fresh, frozen, dried, or freeze-dried berries, or combination thereof.

9. The composition of claim 1, wherein the extract is an ethanol extract, carried out at a temperature of less than 40° C.

10. The composition of claim 1, comprising one or more of cyanidin, delphinidin, epicatechin gallate or epigallocatechin gallate.

11. The composition of claim 1, further comprising a pharmaceutically acceptable carrier or excipient.

12. A method for making a polyphenol-rich extract, comprising:
1. mixing 50-70% alcohol with a blueberry, bilberry, sloeberry, or chokeberry, to form a mixture;
2. incubating the mixture at room temperature for a time ranging from 1 hour to 90 days while rotating daily, to form an incubated mixture;
3. filtering or centrifuging solid matter out of the incubated mixture, and collecting flow-through of the incubated mixture;
4. optionally, combine the flow-through from Step 3 with a berry powder, to form a second mixture;
5. optionally, incubating the second mixture for 1-3 hours at room temperature, to form a second incubated mixture;
6. optionally, filtering or centrifuging solid matter out of the second incubated mixture and collecting supernatant; and
7. evaporating alcohol under vacuum from the flow-through from step 3 or the supernatant from step 4, at a temperature ≤40° C., to a desired alcohol concentration, such as 20% or less, to form the polyphenol-rich extract.

13. A composition for the treatment of diarrheal disease, comprising at least 0.2% by weight of each of: cyanidin, delphinidin, epicatechin gallate or epigallocatechin gallate, salt thereof, or glycosylate thereof; and a pharmaceutically acceptable carrier or excipient.

14. The composition of claim 13, wherein the weight ratio of cyanidin:delphinidin:epicatechin gallate:epigallocatechin gallate is 300-700:100-30:0.5-2:200-50.

15. The composition of claim 13, wherein the weight ratio of cyanidin:delphinidin:epicatechin gallate:epigallocatechin gallate is 400-600:90-50:0.75-1.5:175-100.

16. The composition of claim 13, wherein the weight ratio of cyanidin:delphinidin:epicatechin gallate:epigallocatechin gallate is 500:70:1:150.

17. The composition of claim 13, wherein each of the epicatechin, epigallocatechin, cyanidin, and delphinidin may independently be in glycosylated form, L-rhamnose, D-glucose, glucorhamnose, galactose, fructose or arabinose form, aglycone form, crystallized form, aqueous solution, alcoholic solution, salt, chloride salt, gallic acid salt, or combination thereof.

18. A method for treating a subject suffering from a diarrheal disease, the method comprising administering the composition of claim 1, to the subject, to treat said subject.

19. A method for treating a subject suffering from a diarrheal disease, the method comprising administering the composition of claim 13, to the subject, to treat said subject.
